(19) United States
(12) Patent Application Publication
(10) Pub. No.: US 2006/0178698 A1
(43) Pub. Date: Aug. 10, 2006

(54) Method and device for canulation and occlusion of uterine arteries

(76) Inventors: Jon T. McIntyre, Newton, MA (US); Michael Madden, Princeton, MA (US)

Correspondence Address: FAY KAPLUN & MARCIN, LLP, 15O BROADWAY, SUITE 702, NEW YORK, NY, 10038, US

(21) Appl. No.: 11/053,343

(22) Filed: Feb. 8, 2005

(51) Int. Cl. A61M 29/00

(52) U.S. Cl. 606/200

(57) ABSTRACT

A method for treating a uterine fibroid comprises forming an incision in a vaginal fornix to expose a first blood vessel supplying the fibroid, forming an opening in the first blood vessel and inserting an introducer into the first blood vessel via the opening in combination with the steps of advancing a catheter to a desired position within the first blood vessel via the introducer and introducing an occlusive agent into the first blood vessel through the catheter to block blood flow through the first blood vessel. A device for treating uterine fibroids comprises an elongated sheath sized for insertion into uterine arteries via an incision in the vaginal fornix, the sheath including a sheath lumen extending from a first sheath opening formed in a proximal end of the sheath to a second sheath opening formed in a distal end of the sheath and a body a distal end of which is connected to the proximal end of the sheath, the body including a body lumen extending therethrough from a first body opening at a proximal end of the body and a second body opening at the distal end thereof, the second body lumen communicating with the sheath lumen in combination with a hemostatic valve controlling the flow of blood through the body lumen.

### FIELD OF INVENTION

The present invention relates generally to methods and devices for canulation and occlusion of uterine arteries for the treatment of fibroids.

### BACKGROUND

Several procedures are available for the treatment of uterine leiomyoma (fibroids) including the delivery of occlusive agents to the uterine arteries to prevent blood flow to the fibroids via catheters introduced into the uterine arteries via access from the femoral artery. However, these methods require a relatively high level of catheterization skill and have, therefore, been primarily performed by interventional radiologists in catheterization labs or angiography suites.

### SUMMARY OF THE INVENTION

The present invention is directed to a method for treating a uterine fibroid comprising forming an incision in a vaginal fornix to expose a first blood vessel supplying the fibroid, forming an opening in the first blood vessel and inserting an introducer into the first blood vessel via the opening in combination with the steps of advancing a catheter to a desired position within the first blood vessel via the introducer and introducing an occlusive agent into the first blood vessel through the catheter to block blood flow through the first blood vessel.

The present invention is further directed to a device for treating uterine fibroids comprising an elongated sheath sized for insertion into a uterine artery via an incision in the vaginal fornix, the sheath including a sheath lumen extending from a first sheath opening formed in a proximal end of the sheath to a second sheath opening formed in a distal end of the sheath and a body a distal end of which is connected to the proximal end of the sheath, the body including a body lumen extending therethrough from a first body opening at a proximal end of the body and a second body opening at the distal end thereof, the second body lumen communicating with the sheath lumen in combination with a hemostatic valve controlling the flow of blood through the body lumen.

### BRIEF DESCRIPTION OF THE DRAWINGS

The accompanying drawings are included to provide a further understanding of the invention and are incorporated in and constitute part of the specification, illustrate several embodiments of the invention and, together with the description, serve to explain examples of the present invention. In the drawings:

FIG. 1 shows a cross-sectional view along a frontal plane of the uterine-vaginal interaction and the spatial relationship of the uterine arteries relative to each;

FIG. 2 shows a front view of the uterine-vaginal interaction.

FIG. 3 shows a sectional view of the introduction of a device into the uterine vessels in accordance with one embodiment of the present method;

FIG. 4 shows a perspective view of a device used to introduce occlusive agents into the uterine vessel in accordance with one embodiment of the present method; and

FIG. 5 shows a sectional view of the uterine vessel beneath the incision in the vaginal wall.

FIG. 6 shows a cross-sectional view along a frontal plane of the uterine-vaginal interaction and the introduction of one embodiment of the claimed device into the uterine vessels;

FIG. 7 shows a cross-sectional view of one uterine vessel and the introduction of a catheter with a preformed tip via the sheath of one embodiment of the claimed device.

FIG. 8 shows a cross-sectional view of one uterine vessel and the deployment of embolic particles therein;

FIG. 9 shows a cross-sectional view of one uterine vessel and the deployment of an umbrella-shaped device therein;

FIG. 10 shows a cross-sectional view of one uterine vessel and the deployment of suture material;

FIG. 11 shows a cross-sectional view of one uterine vessel and the deployment of vascular plugs; and

FIG. 12 shows a kit for use in carrying out the claimed method.

### DETAILED DESCRIPTION

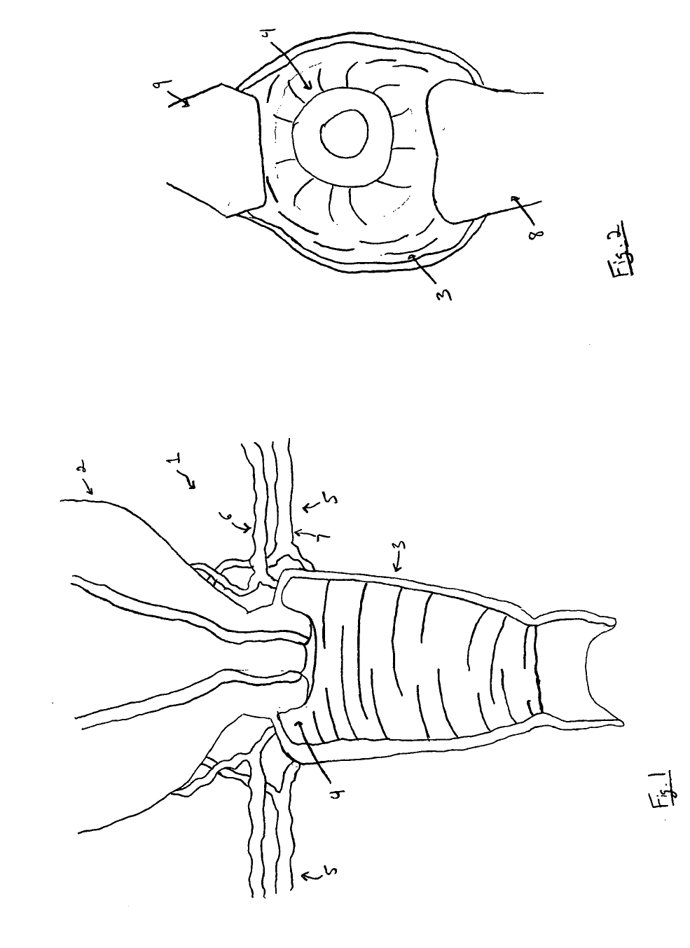

The present invention is directed to a simplified method and device for treating fibroids without requiring the same high level of catheterization skills required for the prior methods. FIG. 1 shows the structure of the female reproductive system generally seen at 1. The uterus 2 is superior to the vagina 3, with the cervix 4 comprising a lowermost portion of the uterus 2, which connects to the vagina 3. A lowermost portion of the cervix 4 is exposed to the vagina 3. The uterine blood vessels 5, located superior to the vagina 3 and inferior to the uterus 2, include a series of uterine arteries 6 and a series of uterine veins 7.

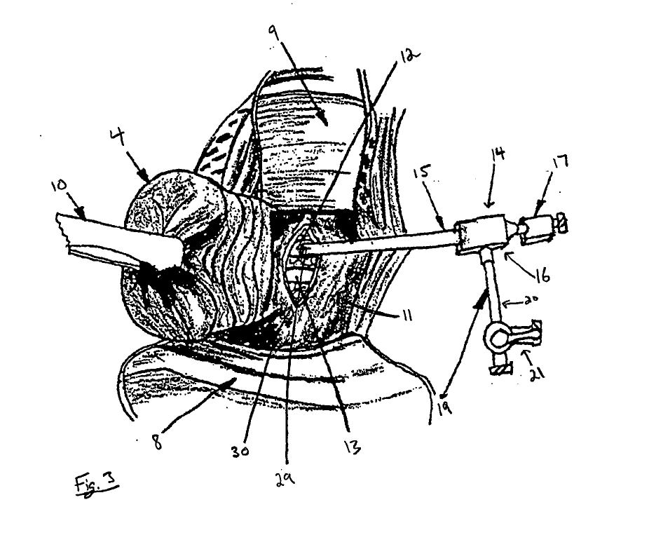

In accordance with the present method, FIG. 1 depicts an initial view of the uterine blood vessels 5. As seen in FIG. 2, visualization of the cervix 4 may be maximized by inserting a speculum 8 into the vagina 3 at a lower position while using a retractor 9 at an upper position of the vagina 3. As would be understood by those of skill in the art, a weighted speculum may be utilized to allow the physician the use of both hands. Additionally, it is preferable that the speculum 8 be inserted at a lowermost portion of the vagina 3, or the six o'clock position with the retractor 9 inserted at an uppermost portion of the vagina 3, or the twelve o'clock position. As would be understood by those skilled in the art, these positions are described as if the physician is viewing the patient from an inferior position along a frontal plane with the patient in the supine position. It is contemplated that a maximized view of the cervix 4 may also be accomplished by means other than the described speculum-retractor combination.

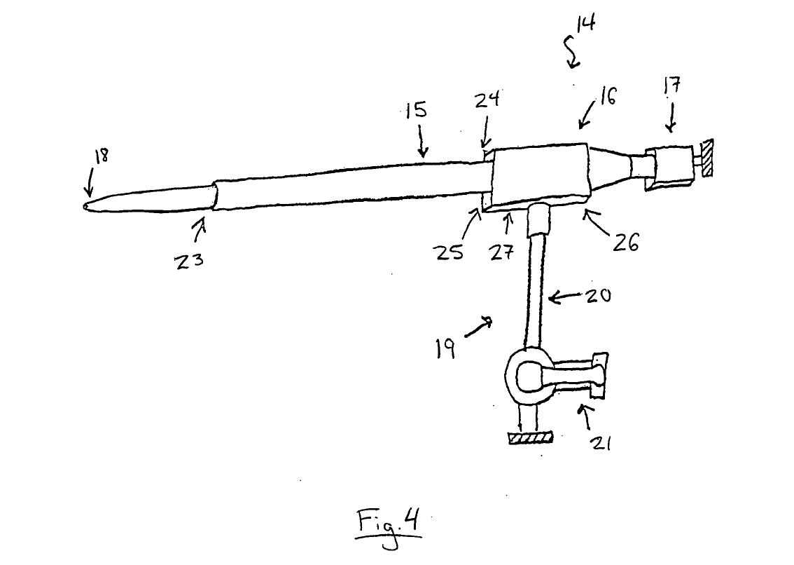

As seen in FIG. 3, the view of the cervix 4 has been maximized by the speculum 8 and the retractor 9. Preferably, the cervix 4 is moved laterally and maintained in a static, off-center position by a clamp 10 or tenaculum to further expose the vaginal fornix 11. However, it is contemplated that the cervix 4 may remain in its original position while the procedure is conducted.

The patient may be placed under general anesthesia, or local anesthesia may be used. For local anesthesia, approximately 1% to 2% lidocaine may be injected intradermally in the vaginal fornix 11 using, for example, a short 25 or 27-gauge needle. It will be understood by those skilled in the art that the procedure will be most effective if performed on the series of uterine arteries 6 on both sides of the uterus 2. However, with some patients, circumstances may require that the procedure be performed on the uterine artery and associated branching 6 on only one side of the uterus 2.

An incision 30 is made in the vaginal fornix 11 to expose a selected uterine artery 12 and a selected uterine vein 13. As would be understood by those skilled in the art, the incision 30 is preferably a partial circumferential incision done with, for example, a surgical blade or an electrocautery bovie unit. Preferably, the incisions 30 are made at the three o'clock and/or 9 o'clock positions relative to the cervix 4.

As would be understood by those skilled in the art, the selected uterine artery 12 must be separated from surrounding tissue to expose this uterine artery 12 to the surface of the operating field. Blunt dissection or any other separation procedure may be used to separate this uterine artery 12 from the surrounding tissue. Blunt dissection may be accomplished with forceps or other medical instrumentation used to remove tissue or fascia. The uterine artery 12 and the uterine vein 13 should then be isolated from adjacent nerves, blood vessels and fascia. Also, it is preferred that the uterine artery 12 and uterine vein 13 be tagged, so as not to be confused with other area vessels or with each other. The tags may be formed, for example, as a loop of suture, surgical tape, umbilical tape, or any other material that will allow the physician easily to identify the vessels.

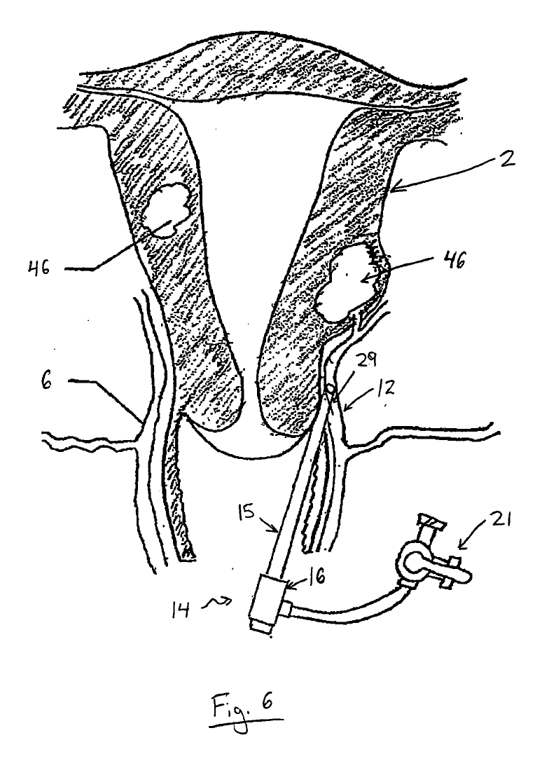

When the uterine artery 12 has been sufficiently cleaned and tagged, a small incision is made in its surface to create an arteriotomy 29. Preferably, the arteriotomy 29 is made transversely using a surgical blade, such as a no. 11 blade. However, it is contemplated that other blade types or incision methods may be used to transect the surface of the uterine artery 12. After the arteriotomy 29 has been formed, flow through the artery may be temporarily stopped by ligation upstream of the arteriotomy 29 using, for example, a clamp, a clip or suturing. For example, FIG. 5 shows bulldog clamps 22 in place upstream of the arteriotomy 29 closing the uterine artery 12 temporarily to prevent blood loss from the arteriotomy 29.

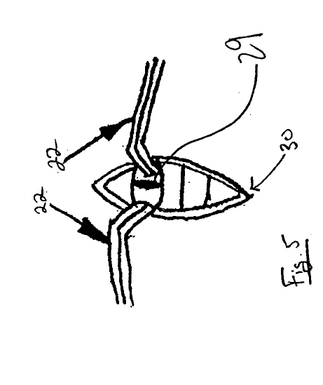

The arteriotomy 29 formed in the uterine artery 12 is preferably made large enough to allow for the introduction of an introducer device 14 thereinto, as shown in FIG. 3. The introducer device 14, shown more clearly in FIG. 4, includes a sheath 15 having a distal end 23 insertable into the uterine artery 12 via the arteriotomy 29. The sheath 15 is preferably formed as a flexible tube, such as a cannula, which may optionally include a trocar on the distal end 23 of the sheath 15. As would be understood by those skilled in the art, the sheath 15 may be any of a variety of sizes and shapes, including an elliptical, circular, rectangular or triangular cross-section, and may have a beveled, rounded or angular distal end 23. The distal end 23 of the sheath 15 may further be pre-shaped to allow an operator to sub-select arterial branches.

As would be understood by those skilled in the art, the sheath 15 may be constructed of a single material, a coextruded composite, or a braid reinforced construction to better transmit torque. The inside diameter of the sheath 15 may be between 3F and 9F but is preferably between 4F and 6F. The sheath 15 is preferably produced of a material with a low coefficient of friction such as Teflon® or polyethylene and may additionally be treated with a lubricous coating for ease of vessel insertion. The lubricous coating may a hydrophilic coating or another coating known to those of ordinary skill in the art. The sheath 15 is preferably made echogenic, either by use of echogenic coatings or compounding agents included directly in the material of the sheath 15, to allow for detection thereof using ultrasound.

At the proximal end of the introducer 14, a hemostasis valve is enclosed within body member 16, which acts as a fluidtight passageway and connection point for the various elements of the introducer device 14. The body member 16 comprises a lumen that communicates with an adjacent lumen of the sheath 15. As such, the body member 16 may be any shape or size sufficient to accommodate the sheath 15 and the other elements of the introducer device 14.

Opposite the distal end 25 of the body member 16, a dilator 17 is removably connected to a proximal end 26 of the body member 16. The dilator 17 preferably includes a distal end 18 tapered or otherwise shaped to provide a smooth insertion into a vessel. The dilator 17 is preferably a tubular member of a diameter less than an inside diameter of the sheath 15. While connected to the introducer 14, the dilator 17 extends longitudinally from the proximal end 26 of body member 16 through the lumen of the body member 16 and the sheath 15. The distal end 18 of the dilator 17 preferably extends beyond the distal end 23 of the sheath 15. Those skilled in the art will recognize that the dilator 17 functions in part to facilitate the ease of insertion of the larger distal end 23 of the sheath 15 into the uterine artery 12.

The body member 16 further includes a hemostatic valve housed within the body member 16 to prevent blood loss through the introducer device 14. The hemostatic valve is preferably manufactured of silicone, latex or another elastomer. The hemostatic valve allows the dilator 17 to be removed while hemostasis is maintained. Multiple devices, including catheters and wires, can be inserted into and removed from the introducer device 14 without the loss of blood. For example, a syringe or other separate device may be used in place of the dilator 17 to detect the return of blood indicating that the distal end 23 is within the uterine artery 12.

A sidearm 19 may be attached to a side surface 27 of the body member 16. The sidearm 19 may comprise a substantially tubular member 20 and a 3-way stopcock 21. The stopcock 21 may be used to divert fluid flow through the sidearm 19 or to occlude flow for a desirable period of time. Preferably, the member 20 is positioned substantially perpendicularly with respect to the dilator 17 and the sheath 15. It is further contemplated that the member 20 may be curved in a desired direction to facilitate introduction of the sheath 15 into the uterus in a position suitable for entry into the artierorotomy. Alternatively, the member 20 may be made flexible so that it may be bent into a desired shape to account for the anatomy of a patient. As would be understood by those skilled in the art, the member 20 may be formed of a compliant material flexible enough to be bent into a desired shape, but with sufficient rigidity that shape is maintained until the member 20 is intentionally bent into a new shape.

Prior to insertion of the sheath 15 into the arteriotomy 29, the introducer device 14 is preferably flushed with saline and the clamps 22 are removed from the portion of the uterine artery 12 into which the dilator 17 and the sheath 15 are to be inserted. A guidewire (e.g., 0.035-0.038 inch in diameter) is first inserted into the arteriotomy 29 toward the uterus 2 to facilitate advancement of the dilator 17 through the uterine artery 12. Alternatively, a Doppler guidewire, such as the 18-gauge Smart-Needle manufactured by CardioVascular Dynamics®, may be used to ensure proper positioning of the introducer device 14 in the uterine artery 12. The obturator of the Smart-Needle contains a Doppler crystal that aims a needle tip toward the center of the arterial lumen based on auditory characteristics of arterial flow. Use of a Doppler guidewire further reduces the level of skill needed to perform the method according to the invention.

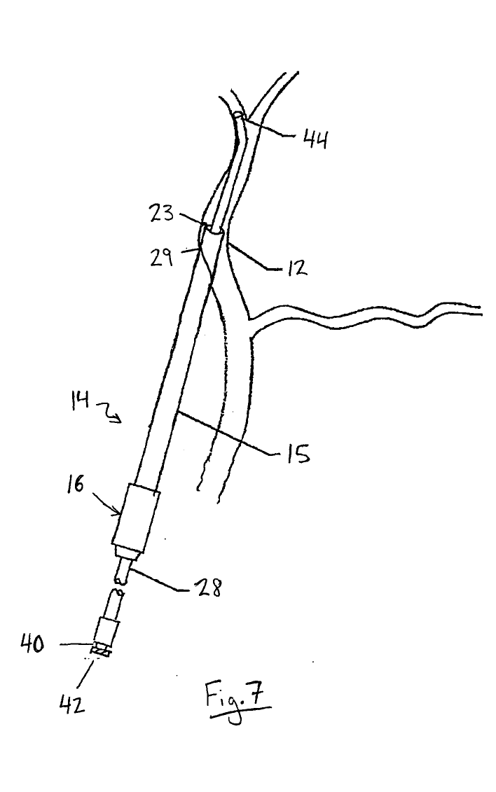

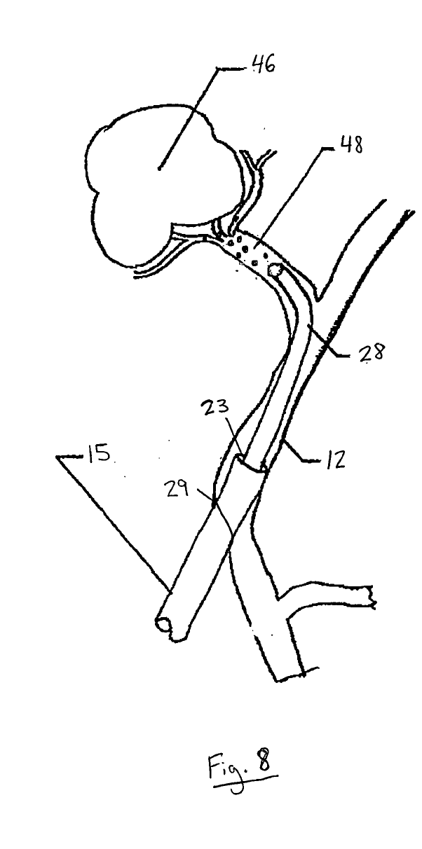

Once the sheath 15 has been inserted into the arteriotomy 29, suction is applied to the dilator 17 to aspirate blood therethrough until there is a free return of blood through the uterine artery 12. As shown in FIG. 6, the dilator 17 may then be removed from the introducer 14. A catheter 28 may then be inserted through the introducer 14, shown in FIG. 7. The catheter 28 is passed through the body member 16 and through the sheath 15 into the uterine artery 12.

The catheter 28 may be constructed of a single layer polymer or may be reinforced with coil or braid and, as would be understood by those skilled in the art, the catheter 28 may comprise polyurethane, pellethane, polyethylene or other known materials. The coil or braid may be comprised of stainless steel, nitinol or any other suitable metals or alloys. The rigidity of the catheter 28 may be substantially constant along its length but preferably varies from the proximal end 42 to a distal tip 44. In one embodiment, the catheter 28 is stiffer at the proximal end 42 to enhance the columnar and torsional strength of this portion and softer toward the distal tip 44 to increase the flexibility of this portion of the catheter 28 allowing it to more easily bend through vessels. The distal tip 44 is preferably made of a soft material such as but not limited to 35D to achieve atraumatic interaction with the vessel walls during catheter positioning.

The catheter 28 includes a lumen for the passage of embolic devices or agents. As shown in FIG. 7, a luer lock fitting 40 is connected to a proximal end 42 of the catheter 28 to accommodate a syringe or other means to inject or aspirate embolic agents or devices through the lumen of the catheter. The catheter 28 may further contain multiple lumens to allow for simultaneous injection of medicines to the uterine artery 12 or for simultaneously deploying multiple embolic devices thereto.

The catheter 28 is passed through the body member 16 and the hemostatic valve therein, through the sheath 15 into the uterine artery 12. The catheter 28 is preferably a microcatheter which may be steered to a desired position within an arterial tree of the uterine artery 12—specifically within the arteries that flow from the uterine artery 12 into the fibroid(s) 46. The catheter 28 may be steerable or deflectable by use of a pullwire or other known means as would be understood by those skilled in the art. In one embodiment, the distal tip 44 of the catheter 28 is preformed to aid in selectively steering to desired vessel branches.

As would be understood by those skilled in the art, guidance of the catheter 28 through the arterial tree may be accomplished through the use of any sufficient imaging method (e.g., color Doppler trans-abdominal ultrasound, etc.). In one embodiment, radiopaque markers or materials may be added to the catheter 28 to enable radiographic or ultrasonic visualization of the position of the catheter 28. Radiographic visualization may be enhanced by use of distal marking bands made from materials including but not limited to, gold, tantalum, tungsten, or by the compounding of materials such as bismuth subcarbonate or barium sulfate directly into the polymer used in production of the catheter 28. Percent loading of such radiopaque materials may be 20% to 40%, but are preferably approximately 30%.

To use ultrasound imaging, the catheter can be constructed from echogenic materials. In this scenario, the imaging method selected will show blood flow to the uterus 2 in real-time, along with the location of the fibroid(s) 46, and the position of the catheter 28 relative to the fibroid(s) 46. This approach allows the physician to guide the catheter 28 without the use of an angiography suite, as required with certain prior methods. Overall, the requirement for advanced catheterization techniques is minimized.

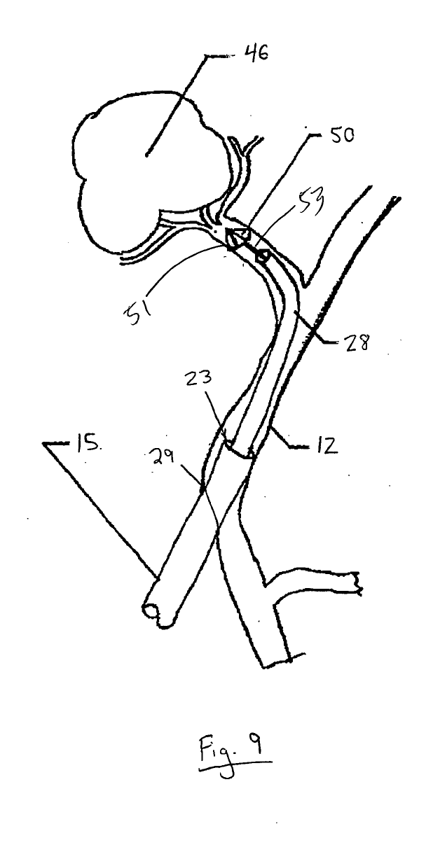

Once the catheter 28 has been positioned in the desired location, blood is aspirated therethrough to remove any air and/or fluid that may have accumulated while the catheter 28 was traveling through the arterial tree. At this point, as shown in FIG. 8, an occlusive agent, for example an embolic agent 48, may be delivered via the catheter 28 to a desired location within the vessels which supply blood to fibroid(s) 46. As would be understood by those skilled in the art, the occlusive agents deployed block blood flow through their respective arteries so that, without nourishment, the fibroid(s) 46 supplied thereby will necrose, while collateral blood flow from unblocked vessels supplies the rest of the uterus.

Preferably, the occlusive agents used in conjunction with this method and device may include, but are not limited to, embolic agents 48 such as polyvinyl alcohol (PVA) particles of varying sizes. The PVA particles, for example, may be those manufactured by Boston Scientific Corp.®, Biosphere®, Cordis®, or other manufacturers. Alternatively, gel foam pieces, polymer plugs, vascular plugs, occluding umbrella-like devices and suture materials may be used as the occlusive agents provided they are capable of extinguishing the flow of blood through the respective vessels. As described above, during injection of the occlusive agents into a blood vessel, the vessel may be temporarily ligated upstream from the arteriotomy 29 by, for example, a clamp 22, a clip, suture or other means to prevent blood loss and to temporarily prevent blood flow through the vessel from disturbing accurate dispersion of the occlusive agents into the uterine artery 12. As would be understood by those skilled in the art, the effectiveness of the embolic agents and termination of blood flow may be determined using ultrasound or any other sufficient imaging modality.

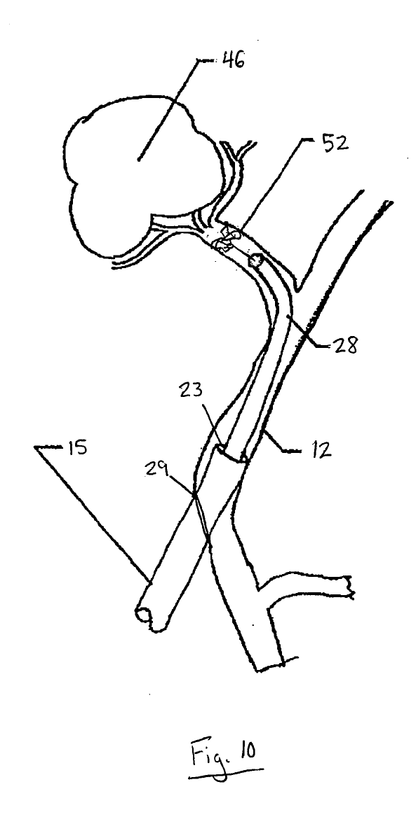

As shown in FIG. 9, the introducer 14 may be used to deploy an umbrella device 50 at a desired location within the uterine artery 12. The umbrella device 50 is detached once in place and left within uterine artery 12 as a permanent or semipermanent implant. The umbrella device 50 includes a tent-like structure 51 coupled to a stem 53. When received within the catheter 28, the umbrella device 50 is collapsed in an insertion configuration in which the tent-like structure 51 is folded against the stem 53 so that a diameter of the umbrella structure is less than an inner diameter of the lumen of the catheter 28 within which it is received. When deployed from the catheter 28, the umbrella device 50 expands to a deployed configuration in which the tent-like structure 51 extends outward away from the stem 53 (e.g., through the action of a biasing member) to a diameter at least as large as an inner diameter of the vessel within which it is deployed. For example, the umbrella device 50 may be inserted through a lumen of the catheter 28 to the distal end thereof by collapsing the tent-like structure 51 and pushing the umbrella device through the lumen using a push-rod which engages the stem 53. When the distal end of the catheter 28 is in the desired position, the umbrella device may be deployed by advancing the push rod until the tent-like structure 52 clears the distal end and expands under the force from the biasing member. Those skilled in the art will recognize that the umbrella device 50 may include a collapsing mechanism if it is desired to make the procedure reversible.

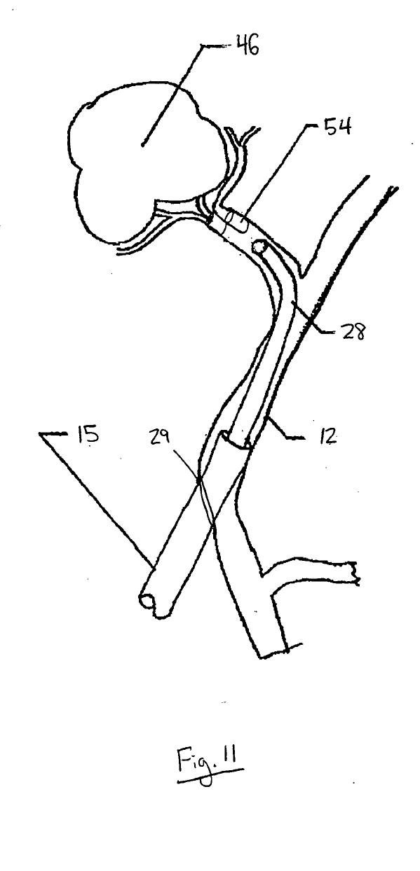

As shown in FIG. 10, the introducer 14 and the present method may be used to deploy a mass of suture material 52 at a selected location within the uterine artery 12. As would be understood by those skilled in the art, the suture material 52 is preferably formed of bio-absorbable materials including but not limited to PGA, PDO, poliglecaprone, polydioxanone, panacryl and caprosyn. The mass of suture material 52 is deployed, for example, by injection through the catheter 28 with a push of saline or radiopaque dye behind the suture mass.

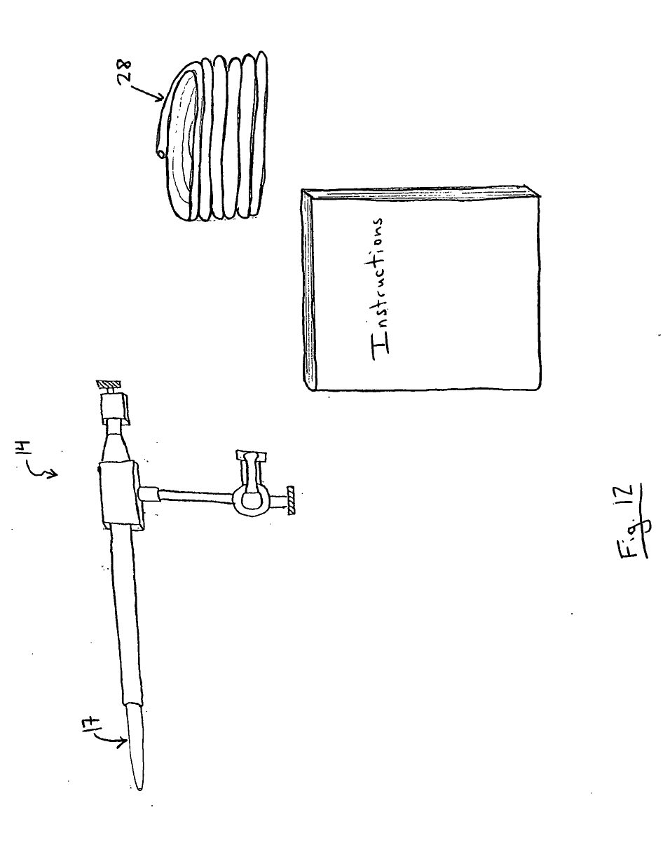

As shown in FIG. 11, the introducer 14 and the present method may be used to deploy vascular plugs 54 via the catheter 28. The vascular plugs 54 are preferably manufactured of materials such as fibrin or collagen. It is further contemplated that the vascular plugs 54 are bio-absorbable. The plugs 54 are preferably guided into the openings of vessels into which they will fit snugly and, as they expand due to the absorption of fluids, they will expand to seal the respective vessels. The plugs 54 may be deployed by graspers, other mechanical means, or by injecting fluid through catheter 28 as described above in regard to the deployment of the mass of suture material 52.

As would be understood by those skilled in the art, the introducer 14 may also be used to deploy vapor into the uterine artery 12 or other vessel(s) to collapse and effectively block the uterine artery 12 or other vessel(s). As would be understood by those of skill in the art, water or saline is introduced as a vapor and , when the vapor condenses, it returns to a very small volume of liquid. This phase change creates a vacuum which collapses the vessel. In addition, the energy generated from the heat of vaporization necroses the artery. The combination of these mechanisms results in an occluded uterine artery.

In a further embodiment of the introducer 14, gel matter may be injected through the catheter 28 into the uterine artery 12. The gel matter may include a lower critical solution temperature (LCST) material such as the LCST material disclosed in U.S. Pat. No. 6,664,594, the entire disclosure of which is herein expressly incorporated by reference herein. LCST material is injected in the uterine artery 12 via the catheter 28 in a liquid state at a temperature below body temperature. Then, as the material is warmed above the critical solution temperature by the warmth of the body, the LCST material changes phase to a gel blocking the uterine artery 12 or other vessel to block off blood flow to the fibroid 46 as described above. This embodiment allows for restoration of blood flow through the uterine artery or other vessel after treatment has been completed. Specifically, this may be accomplished by cooling the LCST material (e.g., by injecting material colder than the critical solution temperature into the vessel via a first lumen of a removal device to re-liquefy the LCST material which is then removed through a second lumen of the removal device under suction. Thus, after the fibroid has been starved off through depletion of its blood flow, flow to down stream tissues is restored by removing the blockage.

Additional embodiments of the introducer 14 may deploy gel foam pieces, polymer plugs, and occlusion balloons. Alternatively, as would be understood by those skilled in the art, an occlusion balloon may be inflated within the uterine artery 12 to occlude flow to the fibroid 46 or other occlusive agents capable of extinguishing blood flow through the respective vessels may also be used.

Upon verification that blood flow has been terminated in the desired vessels, the catheter 12 may be removed and the arteriotomy 29 and incision 30 in the vaginal fornix 11 may be closed. As would be understood by those skilled in the art, each incision may be closed, for example, with a running or continuous stitch. After the incisions have been closed, the procedure may be repeated if necessary on the laterally opposite side of the uterus 2, beginning with an incision in the nine o'clock position relative to the cervix 4. For most patients, it will be necessary to perform the procedure bilaterally.

An additional embodiment of the present method involves a percutaneous location of the uterine artery 12 by palpation. Upon location of the vessel, lidocaine may be applied to the area, and a skin puncture made over the vessel. The puncture may be done with, e.g., a no. 11 blade. A needle may then be advanced toward the uterine artery 12. Preferably, an 18-gauge Seldinger or “single wall puncture” needle inserted at about a 45 degree angle relative to the uterine artery 12 when the patient is oriented in a supine position.

Once the needle has transfixed the artery 12, the obturator of the needle may be removed and replaced with a syringe, which may be a fluid-filled 100 cc syringe. As would be understood by those skilled in the art, the location of the syringe in the blood vessel may be confirmed by aspirating blood therefrom. The syringe may then be removed to allow a guidewire to be advanced through the needle into the artery. Once the guidewire is in place, the needle may be removed over the guidewire.

The guidewire allows the physician to guide the sheath 15 of the introducer device 14 into the uterine artery 12. In this embodiment of the method, the physician may desire use of the sidearm 19 of the introducer device 14, as insertion of the sheath 15 into the uterine artery 12 may be facilitated by rotating the introducer device 14 as it progresses through the soft tissue. Once the sheath 15 of the introducer device 14 has been fed into the uterine artery 12, the dilator 17 on the introducer device 14, which acts in the same manner as described above, is replaced by a catheter 28 for the delivery of occlusive agents into the uterine artery 12 in the same manner as previously described. In a further embodiment, the uterine artery 12 may be accessed through laparoscopic surgery via a trocar penetrating the abdomen. One or more of the introducer device 14 and the catheter 28 may then be fed through the trocar using the introducer device 14 as previously described.

The device of the present invention may additionally be assembled together as a kit for the treating of uterine fibroids or other tissues in accordance with any or all of the methods described above. An exemplary embodiment of such a kit is shown in FIG. 12 including a device 14, a catheter 28 and instructions for performing all of the methods described herein.

It will be apparent to those skilled in the art that various modifications and variations can be made in the structure and the methodology of the present invention, without departing from the spirit or scope of the invention. Thus, it is intended that the present invention cover the modifications and variations of this invention provided they come within the scope of the appended claims and their equivalents.

What is claimed is:

1. A method for treating a uterine fibroid comprising:
forming an incision in a vaginal fornix to expose a first blood vessel supplying the fibroid;
forming an opening in the first blood vessel;
inserting an introducer into the first blood vessel via the opening;
advancing a catheter to a desired position within the first blood vessel via the introducer; and
introducing an occlusive agent into the first blood vessel through the catheter to block blood flow through the first blood vessel.

2. The method according to claim 1, wherein the occlusive agent is an embolic agent.

3. The method according to claim 1, wherein the occlusive agent includes an LCST material with a critical temperature below body temperature, and introducing the occlusive agent into the first blood vessel includes the substeps of maintaining the temperature of the LCST material below the critical temperature prior to introduction into the first blood vessel.

4. The method according to claim 2, wherein the embolic agent includes one of polyvinyl alcohol particles, a gel foam piece, a polymer plug, a vascular plug, an occluding device and a suture material.

5. The method according to claim 1, wherein the incision in the vaginal fornix is made at a substantially three o'clock position relative to a cervix.

6. The method according to claim 1, wherein the first blood vessel is a uterine artery.

7. The method according to claim 1, further comprising the steps of:
inserting a speculum into a lower portion of a vagina; and, inserting a retractor into an upper portion of the vagina to maximize view of the vaginal fornix.

8. The method according to claim 7, wherein the speculum is a weighted speculum.

9. The method according to claim 7, wherein the speculum is inserted at a lower-most position in the vagina.

10. The method according to claim 7, wherein the retractor is inserted at an uppermost position in the vagina.

11. The method according to claim 1, further comprising the step of tagging the first blood vessel prior to forming an opening therein.

12. The method according to claim 1, wherein the opening in the first blood vessel is formed via a transverse incision.

13. The method according to claim 1, wherein the introducer further comprises a dilator.

14. The method according to claim 13, further comprising the step of aspirating material from the dilator until there is a return of blood from the first blood vessel.

15. The method according to claim 1, wherein the catheter is advanced to the desired position within the first blood vessel using a Doppler ultrasound guidance system.

16. The method according to claim 1, further comprising the step of aspirating material from the catheter to remove air therefrom.

17. The method according to claim 1, further comprising the step of ligating the first blood vessel at a position upstream of a location of the opening to be formed therein.

18. The method according to claim 17, wherein the ligation is performed by one of a clip, a clamp and suture.

19. The method according to claim 1, further comprising the step of repeating the method on a second blood vessel, the second blood vessel supplying blood to one of the fibroid and a second fibroid.

20. The method according to claim 19, wherein the second blood vessel is located on an opposite side of the uterus relative to the first blood vessel, the second blood vessel being accessed by a second incision in the vaginal fornix at a nine o'clock position relative to a cervix.

21. The method according to claim 1, wherein the occlusive agent includes an expandable device.

22. A device for treating uterine fibroids comprising:
an elongated sheath sized for insertion into a uterine artery via an incision in the vaginal fornix, the sheath including a sheath lumen extending from a first sheath opening formed in a proximal end of the sheath to a second sheath opening formed in a distal end of the sheath;
a body a distal end of which is connected to the proximal end of the sheath, the body including a body lumen extending therethrough from a first body opening at a proximal end of the body and a second body opening at the distal end thereof, the second body lumen communicating with the sheath lumen; and
a hemostatic valve controlling the flow of blood through the body lumen.

23. The device according to claim 22, further comprising a catheter sized for passage through the body lumen and the sheath lumen and including a catheter lumen extending therethrough between proximal and distal ends thereof, a flexibility of the catheter varying along its length.

24. The device according to claim 22, further comprising a handle extending proximally from the body so that, when the sheath is in an operative position with a distal end thereof inserted through the vagina into a uterine artery via an incision in the vaginal fornix, the handle remains outside the body.

25. The device according to claim 24, wherein the handle is formed of a compliant material, a flexibility of the compliant material being sufficient to allow a user to bend the handle into a desired shape corresponding to the anatomy of a patient and a rigidity of the compliant material being sufficient to retain the desired shape during use.

26. The device according to claim 25, wherein the handle comprises a tubular member a distal end of which is coupled to the body and a stopcock connected to a proximal end of the tubular member.

27. A system for treating a uterine fibroid comprising:
an insertion device including:
an elongated sheath including a sheath lumen extending from a first sheath opening formed in a proximal end of the sheath to a second sheath opening formed in a distal end of the sheath, the sheath being sized for insertion into a uterine artery supplying blood to the fibroid via an incision in the vaginal fornix; and
a body having a distal end connected to the proximal end of the sheath, the body including a body lumen extending therethrough from a first body opening at a proximal end of the body and a second body opening at the distal end thereof, the second body lumen communicating with the sheath lumen, the body further including a hemostatic valve controlling the flow of blood through the body lumen; and
a catheter sized for insertion through the sheath into the uterine artery.

28. The system according to claim 27, further comprising an occlusive device insertable through the catheter into the uterine artery to block blood flow therethrough.

29. The system according to claim 26, wherein the occlusive device comprises an expandable structure which, when ejected from the catheter, expands to engage the wall of the artery to stop blood flow therethrough.

30. The system according to claim 29, further comprising a flexible push rod insertable through the catheter to eject the expandable structure from the catheter.

31. The system according to claim 29, wherein the expandable structure includes a biasing member biasing the expandable structure toward the expanded state.

32. The system according to claim 28, wherein the occlusive device comprises a balloon coupled to a source of inflation fluid so that, when inflated by the inflation fluid the balloon expands to engage the wall of the artery to stop blood flow therethrough.

33. A kit for treating a uterine fibroid comprising:
an introducer;
a catheter; and
instructions for inserting the catheter into a first blood vessel supplying the fibroid via an incision in the vaginal fornix, advancing the catheter to a desired position within the first blood vessel via the introducer, and introducing an occlusive agent into the first blood vessel through the catheter to block blood flow through the first blood vessel.
